(19) United States
(12) Patent Application Publication
(10) Pub. No.: US 2007/0129611 A1
(43) Pub. Date: Jun. 7, 2007

(54) System, Method and Apparatus for Assessing Menopausal or Post-Hysterectomy Symptoms

(75) Inventors: Anna Ratka, Fort Worth, TX (US); James Simpkins, Fort Worth, TX (US)

Correspondence Address: CHALKER FLORES, LLP, 2711 LBJ FRWY, Suite 1036, DALLAS, TX, 75234, US

(73) Assignee: University of North Texas Health Science Center at Fort Worth, Fort Worth, TX (US)

(21) Appl. No.: 11/567,733

(22) Filed: Dec. 6, 2006

Related U.S. Application Data
(60) Provisional application No. 60/742,818, filed on Dec. 6, 2005.

(51) Int. Cl. A61B 5/00

(52) U.S. Cl. 600/300

(57) ABSTRACT

The present invention provides a system, method and apparatus for assessing a person's menopausal or post-hysterectomy symptoms by providing a first set and a second set of questions to the person, receiving answers to the first set and second set of questions and assessing the person's symptoms based on the received answers. The first set of questions includes four or more questions directed to one or more physical conditions. The second set of questions includes two or more questions directed to one or more medical conditions. The physical conditions may include a duration of the symptoms, a frequency of the symptoms, an episode duration of the symptoms, an intensity of the symptoms, a bothersomeness of the symptoms and an onset age of the symptoms. The medical conditions may include a hysterectomy, a hormone therapy, a non-hormonal therapy and a contraception type.

### FIELD OF THE INVENTION

The present invention relates generally to the field of medical diagnostic and assessment tools and, more particularly, to a system, method and apparatus for assessing menopausal or post-hysterectomy symptoms.

### PRIORITY CLAIM

This patent application is a non-provisional application of U.S. provisional patent application 60/742,818 filed on Dec. 6, 2005 and entitled “System, Method and Apparatus For Assessing Menopausal or Post-Hysterectomy Symptoms,” which is hereby incorporated by reference in its entirety.

### BACKGROUND OF THE INVENTION

The U.S. Census Bureau estimated that the population of women between 45 and 64 years old was 32 million in 2000, and it is estimated that by 2010 it will reach approximately 42 million, and by 2050 it will increase to about 47 million. It is estimated that during the natural course of menopause, approximately 65% to 80% of women experience hot flashes, and 10% to 20% find them nearly intolerable. Thus, during the next five decades, 27 to 37 million American women may experience menopausal hot flashes, and in about 7 million menopausal women these symptoms may be severe and difficult to tolerate. Hot flashes are the biggest complaint among middle-aged women and should be regarded as a public health problem because they affect the health and quality of life of millions of women. Despite their prevalence, hot flashes and their role in women's health are very poorly understood and not well characterized.

Hot flashes can begin during the late premenopausal or early perimenopausal years, but they become more frequent and severe during the late perimenopausal and early postmenopausal years. Hot flashes can last from a few seconds to more than an hour and persist for one year in 95% of affected women and up to five years in 65% of affected women. Hot flashes are not only confined to the menopause associated with natural age-related decline of estrogen and progesterone. Surgical removal of ovaries results in hot flashes in 95% to 100% of oophorectomized women. Hot flashes can be experienced by either women or men treated for breast or prostate cancer with chemotherapy, radiation, selective estrogen receptor modulators (SERM) or gonadotropin-releasing hormones. The incidence and severity of hot flashes has been reported to be influenced by culture, diet, body weight, ethnicity, environmental stressors, and socioeconomic status.

The number of hot flashes and their intensity shows intra- and inter-individual variability over time. Women's perception of hot flashes starts may start with increased blood flow to the extremities, increased heart rate, and anxiety. A flush appears on the face, neck and chest, accompanied with an increase in external skin temperature and external sweating. During hot flashes women frequently suffer from various central nervous system disorders, such as anxiety, problems with attention, reduced learning capacity, difficulty with memory, insomnia, depression, and mood swings. Thus, the occurrence of hot flashes has a powerful negative influence on the quality of life in menopausal women and men.

There are two categories of measures of menopausal vasomotor symptoms: subjective and objective. There is a lack of well-characterized, universally accepted standard instruments to collect self-reported data on the various dimensions of hot flashes. Currently only a limited number of validated instruments for the assessment of menopausal symptoms are available. The Blatt-Kupperman menopausal index developed in the early 1950s, combines self reports with physician ratings, monitors data on the severity of vasomotor symptoms, and was reported to be valid and responsive to change, but was criticized for its many shortcomings. The Women's Health Questionnaire (WHO) is primarily a quality of life instrument with many good qualities, but it has a very limited focus on vasomotor hot flashes. The Menopause Rating Scale (MRS) and the Menopause-Specific Quality of Life (MENQOL) questionnaire are other instruments with a quality of life in focus. These instruments address a variety of climacteric domains, but they provide only minimal assessment of hot flashes, the most common and discomforting vasomotor symptoms of menopause. More specifically, these existing menopausal instruments are primarily designed to capture the characteristics of various climacteric symptoms with an emphasis on somatic, urogenital, and associated symptoms and quality of life, but none of them addresses the in-depth characteristics of hot flashes and thus do not meet the needs of a clinical researcher interested in studies of hot flashes. Moreover, many of these instruments were not validated, and some of them are not readily available.

Currently available instruments for the assessment of hot flashes provide either objective but only momentary measures or subjective but inaccurate and superficial measures of hot flashes. Thus, the existing instruments for the assessment of climacteric symptoms are not specific enough for the measurement of the multiple characteristics of hot flashes. Assessment methods for hot flashes that use self-reported daily diaries or electronic event markers depend heavily on women's adherence, and many of them have not been validated.

Currently, only one objective method is available for assessment of hot flashes and it measures hot flashes by recording external skin conductance. This instrument, the Biolog, measures duration of a single hot flash episode and hot flashes frequency, but do not assess intensity, severity, bothersomeness, or conditions directly related to hot flashes. Due to physical limitations and cost, use of skin-conductance monitors for objective measurement of hot flashes is not convenient.

An ideal hot flashes assessment should include both subjective and objective measures. The use of hot flash diaries and electronic event markers have low sensitivity and seriously underestimate hot flash frequency, intensity and bothersomeness. From the clinical perspective, well measured, standardized, self-reported data on various dimensions of hot flashes may be valuable when used alone or in combination with the objective monitoring devices. As a result, there is a need for a system, method and apparatus for assessing a person's menopausal symptoms that provides a comprehensive, simple, valid, reliable, sensitive, and convenient instrument for subjective assessment of various characteristics of hot flashes.

### SUMMARY OF THE INVENTION

The present invention provides a comprehensive, simple, valid, reliable, sensitive, and convenient instrument for subjective assessment of various characteristics of hot flashes that is convenient, inexpensive and adequate for use in an ambulatory setting. Accordingly, the present invention provides a reliable and simple assessment tool for hot flashes that can assist women and their health care providers in efforts to seek effective treatments for these vasomotor symptoms. The set of subjective and specific measures on multiple dimensions of hot flashes provided by the present invention can serve as a valuable clinical and research tool for measurement of hot flashes and development of effective therapies for discomforting hot flashes experienced by millions of middle-aged women and also men treated for prostate cancer.

The present invention incorporates the use of a Menopausal Vasomotor Symptoms (MVS) survey to provide a comprehensive subjective assessment of hot flashes. This survey was designed specifically for in-depth, brief, and standardized subjective assessment of multiple dimensions of hot flashes. The MVS survey is designed to assess multiple dimensions of hot flashes, and to be simple and easy to administer. Hot flashes and associated conditions were addressed with closed-ended questions that required a “Yes” or “No” answer. The MVS survey is designed specifically for the assessment of hot flashes that can be used to: 1) assist in research directed at improving our understanding of the role of hot flashes in during women's aging; 2) assist women and health professionals in efforts to seek effective treatments for these bothersome climacteric symptoms; and 3) provide a reliable clinical tool for developing and testing effective therapies for hot flashes—the discomforting vasomotor symptoms experienced by millions of women.

The survey is sensitive to within-person changes in symptoms of hot flashes, which is a key element in measuring the outcomes of therapies for vasomotor hot flashes occurring during climacteric transition or surgical menopause. Administration of the MVS survey is short, convenient, suitable for ambulatory settings, and very well received by respondents. The MVS survey will be a useful tool in clinical research studies on hot flashes.

There are several areas of clinical research in which the MVS survey can be employed. The MVS survey may play an important role as a guide in evaluating the effectiveness of therapeutic interventions for hot flashes. The MVS survey may also be used to study cross-cultural comparisons of hot flash experiences in different ethnic groups. Since Internet surveys are becoming more popular and have been shown to be reliable in clinical research compared to the paper and pencil survey, an on-line version of the MVS survey can be prepared, validated, and used on large populations of women.

In one study, women experiencing hot flashes took the survey at baseline, and then 14 days, two months, and six months later. Factor analysis was performed. Face and content validity, internal consistency, test-retest reliability, and sensitivity to changes were evaluated. Of the 61 women with hot flashes enrolled initially, 52 (85.2%) completed all study sessions. The MVS survey was found to have good face and content validity and good internal consistency (coefficient α=0.87). Pearson's r coefficients at the 14-day re-test varied from 0.58 to 1.00. As a results of the analysis, the MVS survey was further refined.

The MVS survey has high face validity, test-retest reliability, and sensitivity to changes in hot flashes over time. Women's subjective answers were reliable and showed very high test-retest correlations. The MVS survey encompasses various specific measures of hot flashes, and the response categories were mutually exclusive. The administration of the survey in person (face-to-face) guaranteed that 100% of the responses would be completed, providing more confidence in the results and assured authenticity of the self-reported information. The survey required answers that were simple and very specific. It was sufficient to find out whether a study participant agreed with the question. The return rate was high; 85.2% of the women completed all three sessions administered during six months. The success of the participation in this study could be attributed to user-friendly questions that were closed-response, short, specific, stated in a neutral manner, and did not require the respondent to write down the answers. The format of the MVS survey and its concise, one-page layout made participating women more willing to answer the questions. The MVS survey was suitable and convenient for use in an ambulatory setting. The MVS survey provides a comprehensive measure of not just one hot flash, but of the entire hot flash syndrome. The MVS survey can serve as a valid, reliable and convenient assessment tool with the power to capture a variety of self-reported characteristics of hot flashes that change over time.

The present invention provides a method of assessing a person's menopausal or post-hysterectomy symptoms by providing a first set and a second set of questions to the person, receiving answers to the first set and second set of questions and assessing the person's menopausal or post-hysterectomy symptoms based on the received answers. The first set of questions includes four or more questions directed to one or more physical conditions of the person. The second set of questions includes two or more questions directed to one or more medical conditions of the person. In addition, the present invention can be implemented as a computer program embodied on a computer readable medium wherein the steps are performed by executing one or more code segments.

The physical conditions may include a duration of the menopausal or post-hysterectomy symptoms, a frequency of the menopausal or post-hysterectomy symptoms, an episode duration of the menopausal or post-hysterectomy symptoms, an intensity of the menopausal or post-hysterectomy symptoms, a bothersomeness of the menopausal or post-hysterectomy symptoms and an onset age of the menopausal or post-hysterectomy symptoms. The medical conditions may include a hysterectomy, a hormone therapy, a non-hormonal therapy and a contraception type. The first set of questions may include one or more questions regarding how long the person has had hot flashes, one or more questions regarding how many hot flashes the person has during a day, one or more questions regarding when the person has most of the hot flashes in during a day, one or more questions regarding how long the hot flashes last, one or more questions regarding sweating during the hot flashes, one or more questions regarding interrupted daily activities or sleep and one or more questions regarding an age when the person started having hot flashes. The second set of questions may include one or more questions regarding a hysterectomy, one or more questions regarding hormone therapy for hot flashes, one or more questions regarding herbal preparations for hot flashes and one or more questions regarding contraceptive medications.

The present invention also provides a system for assessing a person's menopausal or post-hysterectomy symptoms that includes a computer and a database communicably coupled to the computer. The computer provides a first set and a second set of questions to the person and receives one or more answers to the first set and second set of questions. The first set of questions includes four or more questions directed to one or more physical conditions of the person. The second set of questions includes two or more questions directed to one or more medical conditions. The one or more answers to the first set and second set of questions are used to assess the person's menopausal or post-hysterectomy symptoms. The database stores the first set and the second set of questions and the one or more answers to the first set and second set of questions. The present invention may also include a communications network communicably coupled to the computer and one or more additional computers communicable coupled to the network that provide access to the database to one or more medical entities (e.g., a hospital, a clinic, a doctor's office, an educational facility, a governmental medical facility, a doctor, a researcher, etc.). The computer or the one or more additional computers may include a personal computer, a laptop computer, a personal data assistant, a telephone, a mobile telephone or a wireless communication device.

The present invention is described in detail below with reference to the accompanying drawings.

### BRIEF DESCRIPTION OF THE DRAWINGS

The above and further advantages of the invention may be better understood by referring to the following description in conjunction with the accompanying drawings, in which:

FIG. 1 is a diagram illustrating a system for assessing a person's menopausal or post-hysterectomy symptoms in accordance with one embodiment of the present invention;

FIG. 2 is a flow chart illustrating a method for assessing a person's menopausal or post-hysterectomy symptoms in accordance with one embodiment of the present invention;

FIG. 3 is a Menopausal Vasomotor Symptoms (MVS) survey in accordance with one embodiment of the present invention;

FIG. 4 is a MVS survey in accordance with another embodiment of the present invention;

FIG. 5 is a flow chart illustrating a method for assessing a person's menopausal or post-hysterectomy symptoms in accordance with another embodiment of the present invention;

FIG. 6 is a flow chart illustrating a method for assessing a person's menopausal or post-hysterectomy symptoms in accordance with another embodiment of the present invention; and

FIG. 7 is a flow chart illustrating a method for assessing a person's menopausal or post-hysterectomy symptoms in accordance with another embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. The discussion herein relates primarily to the assessment of a person's menopausal or post-hysterectomy symptoms, but it will be understood that the concepts of the present invention are applicable to survey-based medical diagnosis and assessment.

The present invention provides a comprehensive, simple, valid, reliable, sensitive, and convenient instrument for subjective assessment of various characteristics of hot flashes that is convenient, inexpensive and adequate for use in an ambulatory setting. Accordingly, the present invention provides a reliable and simple assessment tool for hot flashes that can assist women and their health care providers in efforts to seek effective treatments for these vasomotor symptoms. The set of subjective and specific measures on multiple dimensions of hot flashes provided by the present invention can serve as a valuable clinical and research tool for measurement of hot flashes development of effective therapies for discomforting hot flashes experienced by millions of middle-aged women and also men treated for prostate cancer.

The present invention incorporates the use of a Menopausal Vasomotor Symptoms (MVS) survey to provide a comprehensive subjective assessment of hot flashes. This survey was designed specifically for in-depth, brief, and standardized subjective assessment of multiple dimensions of hot flashes. The MVS survey is designed to assess multiple dimensions of hot flashes, and to be simple and easy to administer. Hot flashes and associated conditions were addressed with closed-ended questions that required a “Yes” or “No” answer. The MVS survey is designed specifically for the assessment of hot flashes that can be used to: 1) assist in research directed at improving our understanding of the role of hot flashes in during women's aging; 2) assist women and health professionals in efforts to seek effective treatments for these bothersome climacteric symptoms; and 3) provide a reliable clinical tool for developing and testing effective therapies for hot flashes—the discomforting vasomotor symptoms experienced by millions of women.

The survey is sensitive to within-person changes in symptoms of hot flashes, which is a key element in measuring the outcomes of therapies for vasomotor hot flashes occurring during climacteric transition or surgical menopause. Administration of the MVS survey is short, convenient, suitable for ambulatory settings, and very well received by respondents. The MVS survey will be a useful tool in clinical research studies on hot flashes.

There are several areas of clinical research in which the MVS survey can be employed. The MVS survey may play an important role as a guide in evaluating the effectiveness of therapeutic interventions for hot flashes. The MVS survey may also be used to study cross-cultural comparisons of hot flash experiences in different ethnic groups. Since Internet surveys are becoming more popular and have been shown to be reliable in clinical research compared to the paper and pencil survey, an on-line version of the MVS survey can be prepared, validated, and used on large populations of women.

In one study, women experiencing hot flashes took the survey at baseline, and then 14 days, two months, and six months later. Factor analysis was performed. Face and content validity, internal consistency, test-retest reliability, and sensitivity to changes were evaluated. Of the 61 women with hot flashes enrolled initially, 52 (85.2%) completed all study sessions. The MVS survey was found to have good face and content validity and good internal consistency (coefficient α=0.87). Pearson's r coefficients at the 14-day re-test varied from 0.58 to 1.00. As a results of the analysis, the MVS survey was further refined.

The MVS survey has high face validity, test-retest reliability, and sensitivity to changes in hot flashes over time. Women's subjective answers were reliable and showed very high test-retest correlations. The MVS survey encompasses various specific measures of hot flashes, and the response categories were mutually exclusive. The administration of the survey in person (face-to-face) guaranteed that 100% of the responses would be completed, providing more confidence in the results and assured authenticity of the self-reported information. The survey required answers that were simple and very specific. It was sufficient to find out whether a study participant agreed with the question. The return rate was high; 85.2% of the women completed all three sessions administered during six months. The success of the participation in this study could be attributed to user-friendly questions that were closed-response, short, specific, stated in a neutral manner, and did not require the respondent to write down the answers. The format of the MVS survey and its concise, one-page layout made participating women more willing to answer the questions. The MVS survey was suitable and convenient for use in an ambulatory setting. The MVS survey provides a comprehensive measure of not just one hot flash, but of the entire hot flash syndrome. The MVS survey can serve as a valid, reliable and convenient assessment tool with the power to capture a variety of self-reported characteristics of hot flashes that change over time.

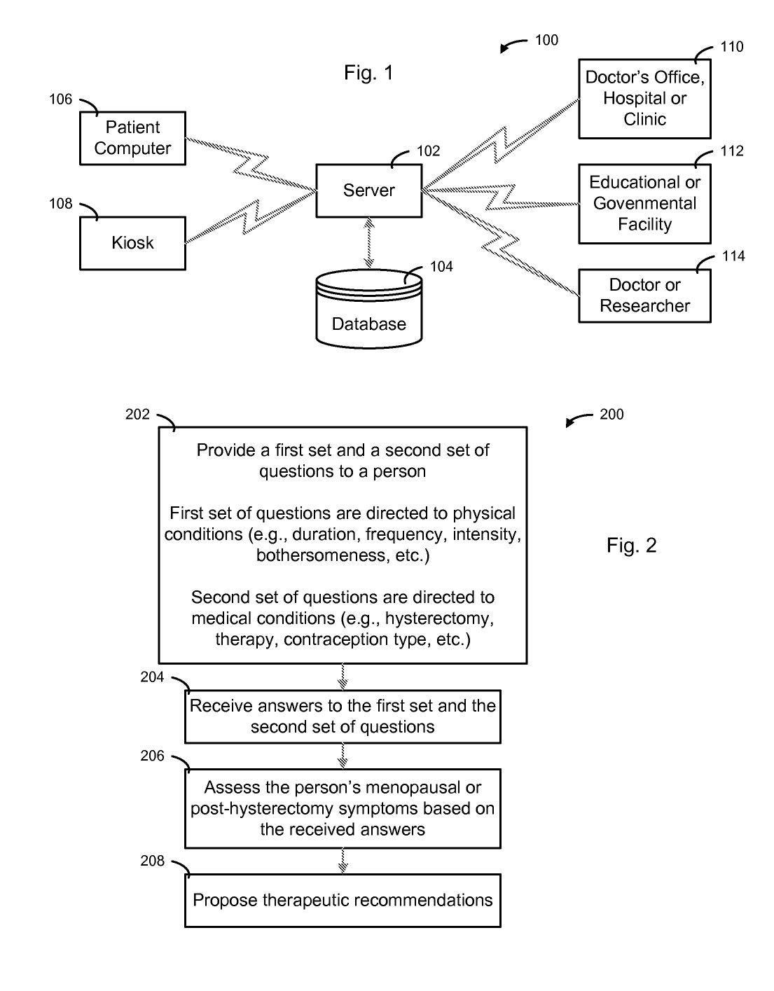

Now referring to FIG. 1, a diagram illustrating a system 100 for assessing a person's menopausal or post-hysterectomy symptoms (e.g., hot flashes, etc.) in accordance with one embodiment of the present invention is shown. System 100 includes a computer 102 and a database 104 communicably coupled to the computer 102. The computer 102 provides a first set and a second set of questions to the person and receives one or more answers to the first set and second set of questions. The one or more answers to the first set and second set of questions are used to assess the person's menopausal or post-hysterectomy symptoms. The database 104 stores the first set and the second set of questions and the one or more answers to the first set and second set of questions. The database 104 can be internal or external to computer 102 and can be physically or wirelessly connected to computer 102 or connected via a communications network (e.g., local area network, wide area network, Internet, satellite, cellular, etc.). Moreover, the database 104 can be specific to the person, a health care provider, a company, a specific location (e.g., clinic, hospital, doctor's office, etc.), a geographic area (e.g., city, county, region, state, nation, world, etc.), a study or test group, a clinical trial, etc.

The person can be presented with the questions and input her/his answers directly into computer 102 or the MVS survey can be administered by a health care provider, who then inputs the answers into computer 102 for analysis and storage in database 104. Computer 102 can be a server computer (as shown) that is communicably coupled to one or more additional computers, such as a patient computer 106, a kiosk 108 or one or more medical entities (e.g., a doctor's office, hospital or clinic 110, an educational or governmental facility 112, a doctor or researcher 114, etc.). The computer 102 or the one or more additional computers (106, 108, 110, 112 or 114) may include a personal computer, a laptop computer, a personal data assistant, a telephone, a mobile telephone, a wireless communication device or any other suitable input/output device. The same system 100 can be used to reassess the person's menopausal or post-hysterectomy symptoms after a specified time period (e.g., approximately fourteen days, two months, six months, etc.). The reassessment is performed by providing the first set and the second set of questions to the person after the specified time period, receiving the answers to the first and second sets of questions and assessing the person's menopausal or post-hysterectomy symptoms based on the received answers. The initial assessment and/or reassessment can be used to determine a treatment for the menopausal or post-hysterectomy symptoms based on the assessment and/or evaluate the effectiveness of the treatment.

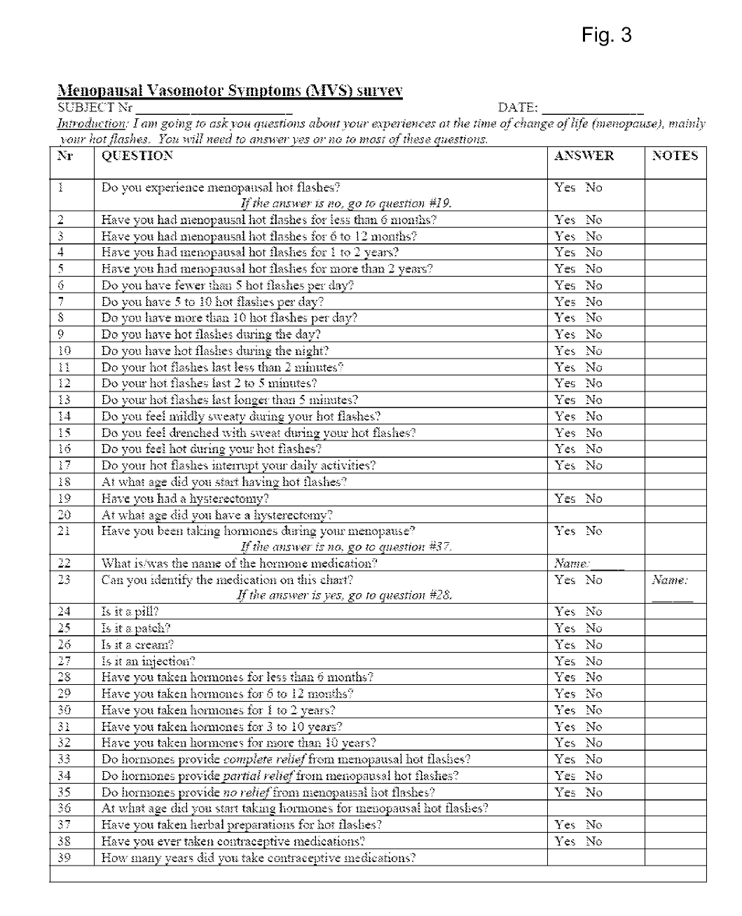

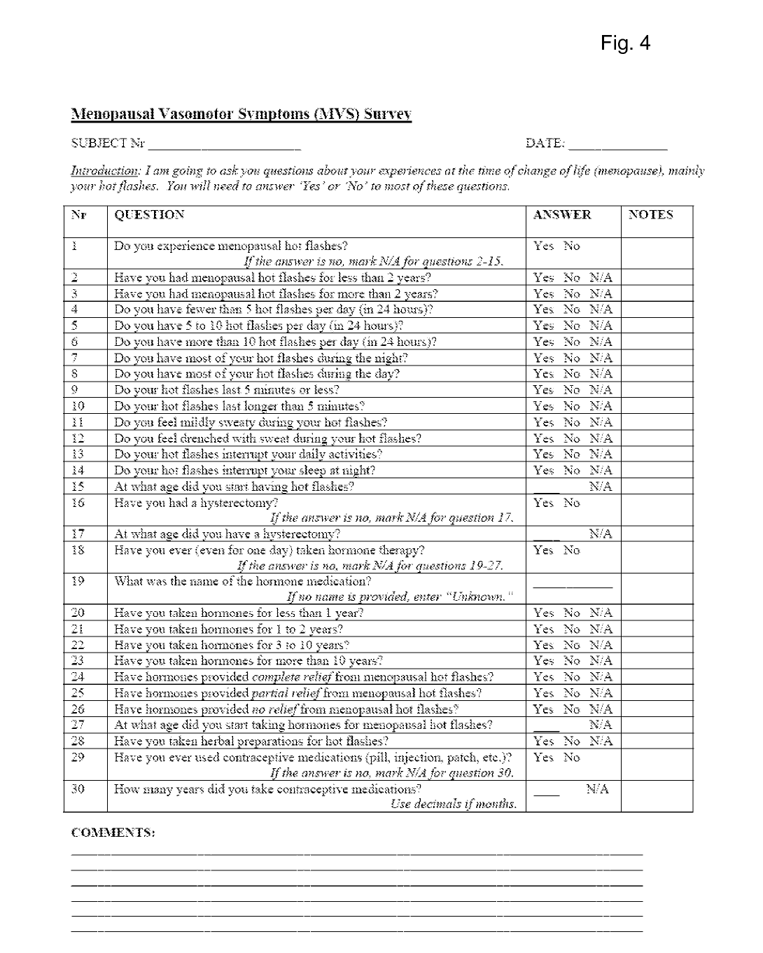

The first set of questions includes four or more questions directed to one or more physical conditions of the person. The physical conditions may include a duration of the menopausal or post-hysterectomy symptoms, a frequency of the menopausal or post-hysterectomy symptoms, an episode duration of the menopausal or post-hysterectomy symptoms, an intensity of the menopausal or post-hysterectomy symptoms, a bothersomeness of the menopausal or post-hysterectomy symptoms and an onset age of the menopausal or post-hysterectomy symptoms. The second set of questions includes two or more questions directed to one or more medical conditions. The medical conditions may include a hysterectomy, a hormone therapy, a non-hormonal therapy and a contraception type. For example, the first set of questions may include one or more questions regarding how long the person has had hot flashes, one or more questions regarding how many hot flashes the person has during a day, one or more questions regarding when the person has most of the hot flashes in during a day, one or more questions regarding how long the hot flashes last, one or more questions regarding sweating during the hot flashes, one or more questions regarding interrupted daily activities or sleep and one or more questions regarding an age when the person started having hot flashes. The second set of questions may include one or more questions regarding a hysterectomy, one or more questions regarding hormone therapy, one or more questions regarding herbal preparations and one or more questions regarding contraceptive medications. The questions that form the MVS survey (first set and second set of questions) will be described in more detail in reference to FIGS. 3 and 4.

Referring now to FIG. 2, a flow chart illustrating a method 200 for assessing a person's menopausal or post-hysterectomy symptoms (e.g., hot flashes) in accordance with one embodiment of the present invention is shown. A first set and a second set of questions are provided to the person in block 202. The first set of questions includes four or more questions directed to one or more physical conditions of the person. The second set of questions includes two or more questions directed to one or more medical conditions of the person. The person's answers to the first set and second set of questions are received in block 204. The answers will typically be stored in a database, which can be accessed by one or more medical entities, such as a hospital, a clinic, a doctor's office, an educational facility, a governmental facility, a doctor or a researcher. Thereafter, the person's menopausal or post-hysterectomy symptoms are assessed based on the received answers in block 206. The assessment may involve comparing the received answers to one or more diagnostic profiles, one or more baseline criteria, a stored library of previously received answers and corresponding patient profiles. This comparison can be performed automatically, manually or combination thereof The assessment can be then be provided to the person electronically, in writing or in person. Furthermore, this person-specific assessment can be used to make recommendations for an optimal and individualized therapy for hot flashes in block 208.

The method 200 can be repeated multiple times for a particular person to reassess the person's menopausal or post-hysterectomy symptoms after a specified time period (e.g., approximately fourteen days, two months, six months, etc.). The reassessment is performed by providing the first set and the second set of questions to the person after the specified time period (block 202), receiving the answers to the first and second sets of questions (block 204) and assessing the person's menopausal or post-hysterectomy symptoms based on the received answers (block 206). The initial assessment and/or reassessment can be used to determine a treatment for the menopausal or post-hysterectomy symptoms based on the assessment and/or evaluate the effectiveness of the treatment. In addition, a third set of questions can be provided to the person in order to create a profile for the person and/or verify the identity of the person. The method 200 may also include receiving one or more diary entries from the person and storing the one more diary entries; or receiving medical information about the person from a health care provider (e.g., a doctor, a nurse, a physician's assistant, a researcher, a medical technician, etc.) and storing the medical information. Note that the present invention can be implemented as a computer program embodied on a computer readable medium wherein the steps are performed by executing one or more code segments.

Now referring to FIG. 3, a Menopausal Vasomotor Symptoms (MVS) survey in accordance with one embodiment of the present invention is shown. The survey includes a first set of questions that includes four or more questions directed to one or more physical conditions of the person (questions 1-18) and a second set of questions that includes two or more questions directed to one or more medical conditions of the person (questions 19-39). The physical conditions may include a duration of the menopausal or post-hysterectomy symptoms (questions 1-5), a frequency of the menopausal or post-hysterectomy symptoms (questions 6-10), an episode duration of the menopausal or post-hysterectomy symptoms (questions 11-13), an intensity of the menopausal or post-hysterectomy symptoms (questions 14-16), a bothersomeness of the menopausal or post-hysterectomy symptom (question 17) and an onset age of the menopausal or post-hysterectomy symptoms (question 18). The medical conditions may include a hysterectomy (questions 19-20), a hormone therapy (questions 21-36), a non-hormonal therapy (question 37) and a contraception type (questions 38-39). The first set and second set of questions are substantially all closed-response questions (e.g., a yes, a no or a not applicable).

For example, the first set of questions may include:
one or more questions regarding how long the person has had hot flashes:
   1. Do you experience menopausal hot flashes?
   2. Have you had menopausal hot flashes for less than 6 months?
   3. Have you had menopausal hot flashes for 6 to 12 months?
   4. Have you had menopausal hot flashes for 1 to 2 years?
   5. Have you had menopausal hot flashes for more than 2 years?
one or more questions regarding how many hot flashes the person has during a day:
   6. Do you have fewer than 5 hot flashes per day?
   7. Do you have 5 to 10 hot flashes per day?
   8. Do you have more than 10 hot flashes per day?
one or more questions regarding when the person has most of the hot flashes in during a day:
   9. Do you have hot flashes during the day?
   10. Do you have hot flashes during the night?
one or more questions regarding how long the hot flashes last:
   11. Do your hot flashes last less than 2 minutes?
   12. Do your hot flashes last 2 to 5 minutes?
   13. Do your hot flashes last longer than 5 minutes?
one or more questions regarding sweating during the hot flashes:
   14. Do you feel mildly sweaty during your hot flashes?
   15. Do you feel drenched with sweat during your hot flashes?
one or more questions regarding interrupted daily activities or sleep:
   16. Do you feel hot during your hot flashes?
   17. Do your hot flashes interrupt your daily activities?
one or more questions regarding an age when the person started having hot flashes:
   18. At what age did you start having hot flashes?
      The second set of questions may include:
one or more questions regarding a hysterectomy:
   19. Have you had a hysterectomy?
   20. At what age did you have a hysterectomy?
one or more questions regarding hormone therapy:
   21. Have you been taking hormones during your menopause?
   22. What is/was the name of the hormone medication?
   23. Can you identify the medication on this chart?
   24. Is it a pill?
   25. Is it a patch?
   26. Is it a cream?
   27. Is it an injection?
   28. Have you taken hormones for less than 6 months?
   29. Have you taken hormones for 6 to 12 months?
   30. Have you taken hormones for 1 to 2 years?
   31. Have you taken hormones for 3 to 10 years?
   32. Have you taken hormones for more than 10 years?
   33. Do hormones provide complete relief from menopausal hot flashes?
   34. Do hormones provide partial relief from menopausal hot flashes?
   35. Do hormones provide no relief from menopausal hot flashes?
   36. At what age did you start taking hormones for menopausal hot flashes?
one or more questions regarding herbal preparations:
   37. Have you taken herbal preparations for hot flashes?
one or more questions regarding contraceptive medications:
   38. Have you ever taken contraceptive medications?
   39. How many years did you take contraceptive medications?

Referring now to FIG. 4, a MVS survey in accordance with another embodiment of the present invention is shown. The survey includes a first set of questions that includes four or more questions directed to one or more physical conditions of the person (questions 1-15) and a second set of questions that includes two or more questions directed to one or more medical conditions of the person (questions 16-30). The physical conditions may include a duration of the menopausal or post-hysterectomy symptoms (questions 1-3), a frequency of the menopausal or post-hysterectomy symptoms (questions 4-8), an episode duration of the menopausal or post-hysterectomy symptoms (questions 9-10), an intensity of the menopausal or post-hysterectomy symptoms (questions 11 - 12), a bothersomeness of the menopausal or post-hysterectomy symptom (question 13-14) and an onset age of the menopausal or post-hysterectomy symptoms (question 15). The medical conditions may include a hysterectomy (questions 16-17), a hormone therapy (questions 18-27), a non-hormonal therapy (question 28) and a contraception type (questions 29-30). The first set and second set of questions are substantially all closed-response questions (e.g., a yes, a no or a not applicable).

For example, the first set of questions may include:
one or more questions regarding how long the person has had hot flashes:
   1. Do you experience menopausal hot flashes?
   2. Have you had menopausal hot flashes for less than 2 years?
   3. Have you had menopausal hot flashes for more than 2 years?
one or more questions regarding how many hot flashes the person has during a day:
   4. Do you have fewer than 5 hot flashes per day (in 24 hours)?
   5. Do you have 5 to 10 hot flashes per day (in 24 hours)?
   6. Do you have more than 10 hot flashes per day (in 24 hours)?
one or more questions regarding when the person has most of the hot flashes in during a day:
   7. Do you have most of your hot flashes during the night?
   8. Do you have most of your hot flashes during the day?
one or more questions regarding how long the hot flashes last:
   9. Do your hot flashes last 5 minutes or less?
   10. Do your hot flashes last longer than 5 minutes?
one or more questions regarding sweating during the hot flashes:
   11. Do you feel mildly sweaty during your hot flashes?
   12. Do you feel drenched with sweat during your hot flashes?
one or more questions regarding interrupted daily activities or sleep:
   13. Do your hot flashes interrupt your daily activities?
   14. Do your hot flashes interrupt your sleep at night?
one or more questions regarding an age when the person started having hot flashes:
   15. At what age did you start having hot flashes?
      The second set of questions may include:
one or more questions regarding a hysterectomy:
   16. Have you had a hysterectomy?
   17. At what age did you have a hysterectomy?
one or more questions regarding hormone therapy:
   18. Have you ever (even for one day) taken hormone therapy?
   19. What was the name of the hormone medication?
   20. Have you taken hormones for less than 1 year?
   21. Have you taken hormones for 1 to 2 years?
   22. Have you taken hormones for 3 to 10 years?
   23. Have you taken hormones for more than 10 years?
   24. Have hormones provided complete relief from menopausal hot flashes?
   25. Have hormones provided partial relief from menopausal hot flashes?
   26. Have hormones provided no relief from menopausal hot flashes?
   27. At what age did you start taking hormones for menopausal hot flashes?
one or more questions regarding herbal preparations:
   28. Have you taken herbal preparations for hot flashes?
one or more questions regarding contraceptive medications:
   29. Have you ever used contraceptive medications (pill, injection, patch, etc.)?
   30. How many years did you take contraceptive medications?

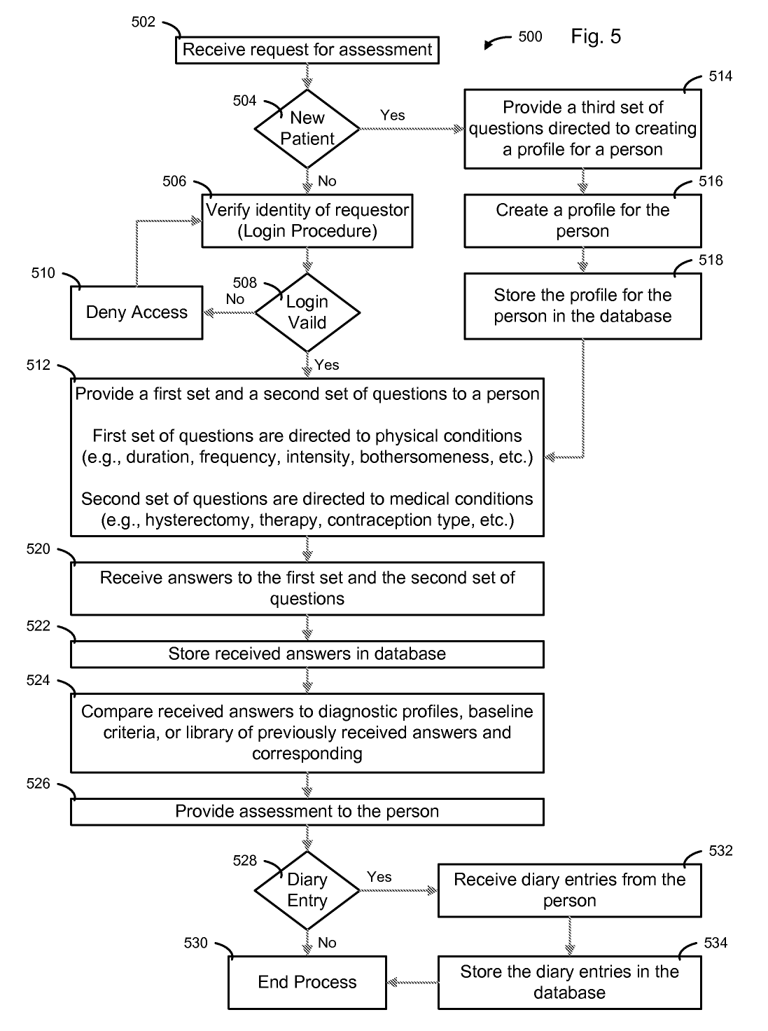

Now referring to FIG. 5, a flow chart illustrating a method 500 for assessing a person's menopausal or post-hysterectomy symptoms in accordance with another embodiment of the present invention is shown. The process begins when a request for assessment is received in block 502. If the request is not from a new patient, as determined in decision block 504, the identity of the requester is verified in block 506 via a login procedure (e.g., user identification and password, biometric authentication, access device, etc.). If the login is not valid, as determined in decision block 508, access is denied in block 510 and the login procedure can be repeated in block 506. Note that there can be a limit on the number of login attempts before the requester is locked out of the system and/or an administrator or security officer is notified. If, however, the login is valid, as determined in decision block 508, a first set and a second set of questions are provided to the person in block 512. As illustrated in FIGS. 3 and 4, the first set of questions includes four or more questions directed to one or more physical conditions of the person and the second set of questions includes two or more questions directed to one or more medical conditions of the person. Note that if the requester is a new patient, as determined in decision block 504, a third set of questions are provided to the person in order to create a demographic profile for the person in block 514. The profile is created in block 516 and stored in a database in block 518. Note that this procedure may include providing the patient with the information or devices to login to the system. Thereafter, a first set and a second set of questions are provided to the person in block 512. Note that this may not occur contemporaneously with the creation of the profile.

The person's answers to the first set and second set of questions are received in block 520 and are stored in a database in block 522. Thereafter, the person's menopausal or post-hysterectomy symptoms are assessed based on the received answers by comparing the received answers to one or more diagnostic profiles, one or more baseline criteria, a stored library of previously received answers and corresponding patient profiles in block 524. Thereafter, the assessment is provided to the person in block 526. Note that this may not occur contemporaneously with the receipt of the answers or assessment, and may involve review by a health care provider. If there is no diary entry, as determined in decision block 528, the process ends in block 530. If, however, there is a diary entry, as determined in decision block 528, one or more diary entries are received from the person in block 532, the one more diary entries are stored in a database in block 534 and the process ends in block 530. Note that method 500 can be repeated multiple times for a particular person to reassess the person's menopausal or post-hysterectomy symptoms after a specified time period (e.g., approximately fourteen days, two months, six months, etc.).

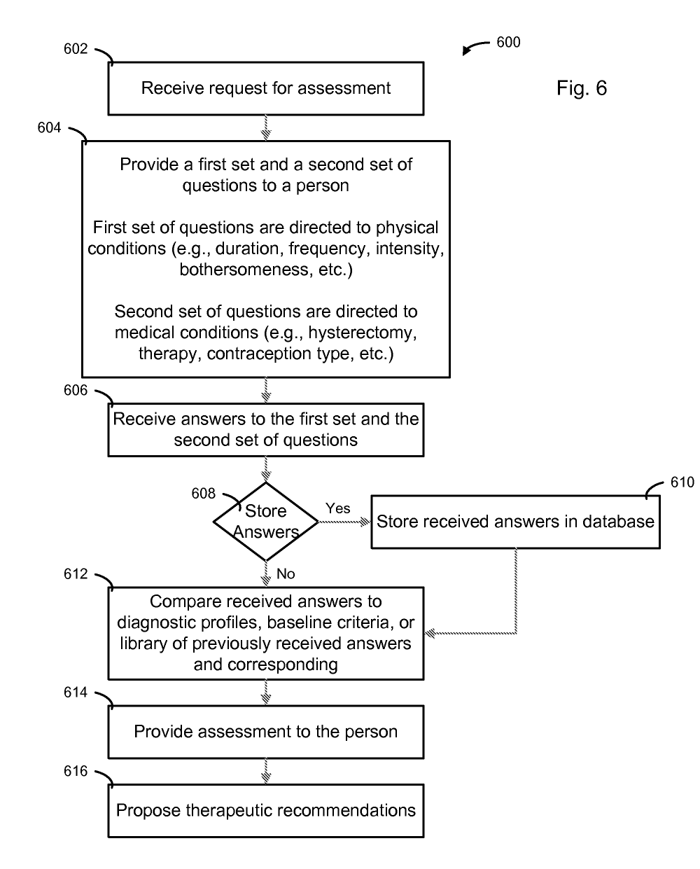

Referring now to FIG. 6, a flow chart illustrating a method 600 for assessing a person's menopausal or post-hysterectomy symptoms in accordance with another embodiment of the present invention is shown. The process 600 begins with a request for assessment is received in block 602. A first set and a second set of questions are provided to the person in block 604. As illustrated in FIGS. 3 and 4, the first set of questions includes four or more questions directed to one or more physical conditions of the person and the second set of questions includes two or more questions directed to one or more medical conditions of the person. The person's answers to the first set and second set of questions are received in block 606. If, the answers are not to be stored (e.g., anonymous Internet or kiosk applications), as determined in decision block 608, the person's menopausal or post-hysterectomy symptoms are assessed in block 612. If, however, the answers are to be stored, as determined in decision block 608, the answers are stored in a database in block 510 and the person's menopausal or post-hysterectomy symptoms are assessed based on the received answers in block 612. The assessment involves comparing the received answers to one or more diagnostic profiles, one or more baseline criteria, a stored library of previously received answers and corresponding patient profiles. Thereafter, the assessment is provided to the person in block 614. One or more therapeutic recommendations may also be proposed in block 616. Note that method 600 can be repeated multiple times for a particular person to reassess the person's menopausal or post-hysterectomy symptoms after a specified time period (e.g., approximately fourteen days, two months, six months, etc.).

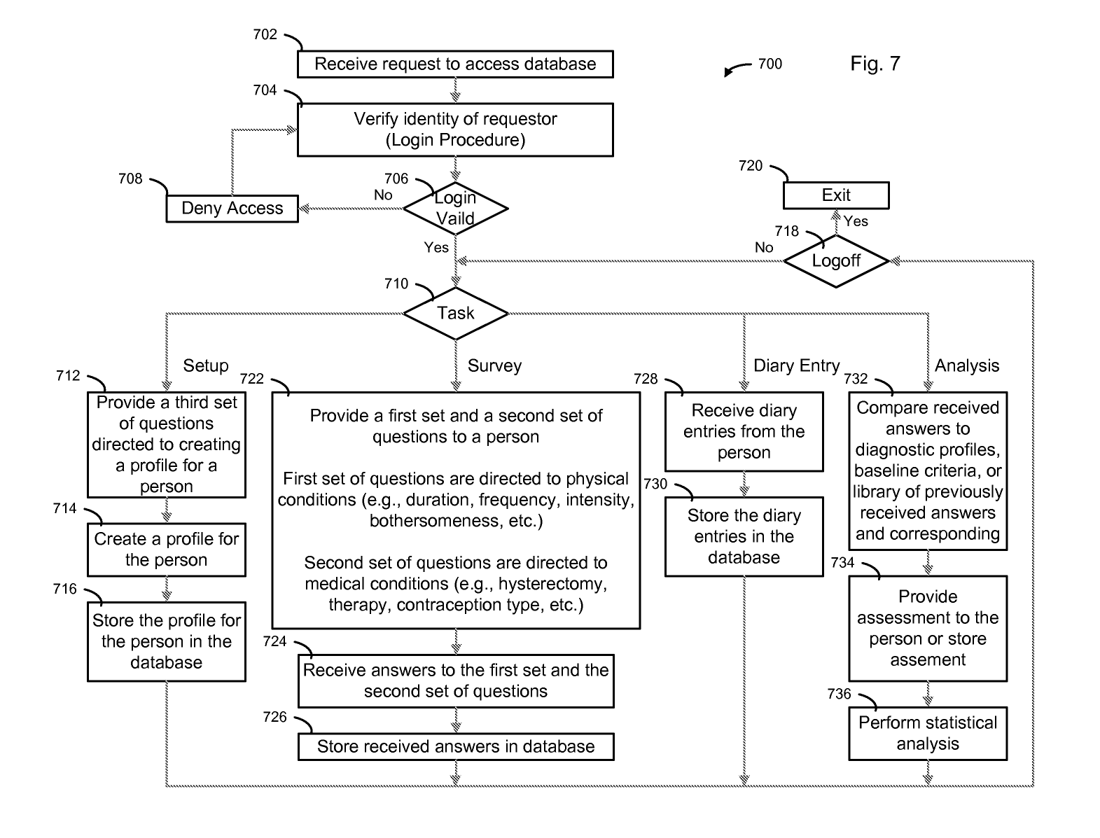

Now referring to FIG. 7, a flow chart illustrating a method 700 for assessing a person's menopausal or post-hysterectomy symptoms in accordance with another embodiment of the present invention is shown. The process begins with a request for access to a database is received in block 702. The requester can be a patient, a person or one or more medical entities, such as a hospital, a clinic, a doctor's office, an educational facility, a governmental facility, a doctor or a researcher. The identity of the requester is verified in block 704 via a login procedure (e.g., user identification and password, biometric authentication, access device, etc.). If the login is not valid, as determined in decision block 706, access is denied in block 708 and the login procedure can be repeated in block 704. Note that there can be a limit on the number of login attempts before the requestor is locked out of the system and/or an administrator or security officer is notified. If, however, the login is valid, as determined in decision block 706, the requestor can perform one or more tasks, which may be limited by a security profile associated with the requestor, as determined in decision block 710. If the requester selects a setup task, as determined in decision block 710, a third set of questions are provided to the person in order to create a profile for the person in block 712. The profile is created in block 714 and stored in a database in block 716. Note that this procedure may include providing the person with the information or devices to login to the system in the future. If the person logs off the system, as determined in decision block 718, the process ends in block 720. If, however, the person does not logoff, as determined in decision block 718, the person can execute another task, as determined in decision block 710.

If the requester selects a survey task, as determined in decision block 710, a first set and a second set of questions are provided to the person in block 722. As illustrated in FIGS. 3 and 4, the first set of questions includes four or more questions directed to one or more physical conditions of the person and the second set of questions includes two or more questions directed to one or more medical conditions of the person. The person's answers to the first set and second set of questions are received in block 724 and are stored in a database in block 726. If the person logs off the system, as determined in decision block 718, the process ends in block 720. If, however, the person does not logoff, as determined in decision block 718, the person can execute another task, as determined in decision block 710.

If the requestor selects a diary entry task, as determined in decision block 710, one or more diary entries are received from the person in block 728 and the one more diary entries are stored in a database in block 730. If the person logs off the system, as determined in decision block 718, the process ends in block 720. If, however, the person does not logoff, as determined in decision block 718, the person can execute another task, as determined in decision block 710.

If the requestor selects an analysis task, as determined in decision block 710, the person's menopausal or post-hysterectomy symptoms are assessed based on the received answers by comparing the received answers to one or more diagnostic profiles, one or more baseline criteria, a stored library of previously received answers and corresponding patient profiles in block 732. Thereafter, the assessment is provided to the person in block 734. Note that this may not occur contemporaneously with the receipt of the answers or assessment, and may involve review by a health care provider. The person may also perform statistical analysis on the database in block 736 depending on the requestor's security profile. Note that method 700 can be repeated multiple times for a particular person to reassess the person's menopausal or post-hysterectomy symptoms after a specified time period (e.g., approximately fourteen days, two months, six months, etc.). If the person logs off the system, as determined in decision block 718, the process ends in block 720. If, however, the person does not logoff, as determined in decision block 718, the person can execute another task, as determined in decision block 710.

The development and testing of the MVS survey will now be discussed. In the approach to the statistical analysis of the MVS survey, the following issues were taken into account: 1) lack of a validated subjective instrument that was adequate in its content to the MVS survey, 2) survey questions within each dimension of hot flashes (duration, frequency, timing, etc) were mutually exclusive, and 3) many dimensions of hot flashes addressed by the survey questions change over time.

Full validity is difficult to establish within a single study. Determination of validity is a continuous process in which evidence needs to be accumulated until construct validity can be assessed. Validity estimates the extent to which a survey measures what it intends to measure. Validity was defined as making common sense and was referred to results that have appearance of truth or reality. Face validity can be based on “appearance” and “common sense.” Content validity is concerned with sample population representativeness and is usually established by content experts. The various dimensions of hot flashes and associated issues are common knowledge not only to the health professionals but most of all to women experiencing hot flashes. The content of the survey questions was sample-oriented and selected exclusively for the population of women with hot flashes. The specific measures of hot-flashes addressed by the survey questions served as a base for evaluation of face and content validity by researchers and clinicians experienced with women affected by the symptoms of hot flashes. With the first set of data collected with the MVS survey, analysis of face and content validity was performed according to the principal methods used to evaluate and compute the validity of study variables, as described above.

A clinical research study was performed to evaluate the validity, reliability, and sensitivity of the Menopausal Vasomotor Symptom (MVS) survey—a new instrument designed for a comprehensive assessment of subjective measures of hot flashes. The results evidence that the MVS survey has a specific content and face validity, has good internal consistency, is reliable and sensitive to within-person changes in hot flashes that occur over time.

The MVS survey was designed to assess in greater depth the vasomotor symptoms that are poorly evaluated with other climacteric scales. The MVS survey encompasses multiple measures of hot flashes, and the response categories are mutually exclusive. The administration of the survey in person guaranteed that 100% of the responses would be completed, providing more confidence in the results. The face-to-face interaction during the MVS survey assured authenticity of the self-reported information. Women's subjective answers were reliable and showed high test-retest correlations. The type of respondents in this study were very pertinent to the research questions included in the MVS survey and helped to make this clinical study more valid. The return rate was high; 85.2% of the women completed all three sessions administered during six months. The success of the participation in this study could be attributed to user-friendly questions that were closed-response, short, specific, stated in a neutral manner, and did not require the respondent to write down the answers. The questions were focused and very specific with high internal consistency. The answers were simple and straight-forward. It was sufficient to find out whether a study participant agreed with the question. The format of the MVS survey and its concise, one-page layout made participating women more willing to answer the questions. An effective follow-up with a reminder letter and a phone call and a reasonable monetary incentive (to compensate for time and travel) may have also contributed to the successful outcome of this project. The MVS survey was suitable and convenient for use in an ambulatory setting. It was noteworthy that most women were remarkably eager to talk about their experiences with hot flashes and inquired about more clinical research on hot flashes.

The qualitative and quantitative dimensions of hot flashes are likely to change over time. The MVS survey was found to be sensitive to changes in symptoms of hot flashes that occurred during a six-month period. The finding that the MVS survey was sensitive to time- related changes in hot flashes added to its face validity. The ability of the MVS survey to detect within-person changes in the symptoms of hot flashes is a key element in measuring the outcomes of therapies for vasomotor hot flashes occurring during climacteric transition or surgical menopause.

During the six months of our clinical study, changes in the symptoms of hot flashes were measured. The natural variations in various dimensions of hot flashes may explain the profound placebo effect (usually downward) observed in many clinical studies that tested treatments for hot flashes. During a 12-month period, hot flash frequency in a placebo group decreased by about 35%. The vasomotor score in a placebo group declined significantly (from 20 to 12) over a period of 24 months. During a 12 week-period, about fourfold decrease in the daily frequency of vasomotor symptoms in placebo-treated women was observed. The significant placebo effect reported in literature may reflect the vulnerability of hot flashes to changes. Thus, hot flashes should be considered as very unstable symptoms. These fluctuations in hot flashes can be evoked by endogenous (mostly unknown) or exogenous factors. Time-related changes in the qualitative and quantitative characteristics of hot flashes assessed with the MVS survey during the three study sessions performed in this project will be presented separately.

The MVS survey was specifically designed to be a brief and standardized measure of various dimensions of hot flashes, to be used for comparative purposes in different clinical studies either alone or in conjunction with objective instrument measures. This survey provides a comprehensive measure of not just one hot flash, but of the entire hot flash syndrome as it is perceived by a woman. This survey appears to have high internal consistency. Administration of the MVS survey provided reliable information—no missing data, no salient measurement error, and sensitivity retained over time. The MVS survey can serve as a convenient assessment tool with the power to capture a variety of self-reported characteristics of hot flashes. The accuracy to which the refined MVS survey can measure hot flashes will be investigated in a separate study in which the self-reported results of the MVS survey will be compared with objective measures of hot flashes. In clinical research, the MVS survey may play an important role as a guide in evaluating the effectiveness of therapeutic interventions for hot flashes. The MVS survey may also be used to study cross-cultural comparisons of hot flash experiences in different ethnic groups. Becasue Internet surveys are becoming more popular and have been shown to be reliable in clinical research compared to the paper and pencil survey, the MVS survey can be expanded to an on-line version.

In conclusion, the MVS survey provided an in-depth assessment of the multiple dimensions of hot flashes with good internal consistency. The survey was shown to be reliable and sensitive to within-person changes in symptoms of hot flashes. Administration of the MVS survey was brief, convenient, and suitable for an ambulatory setting. The MVS survey has the potential to becoming a useful tool for assessing multiple self-reported dimensions of hot flashes and can serve as a guide to therapeutic interventions for hot flashes.

Healthy women between 40 and 58 years old were recruited for this clinical study. The main inclusion criterion was current experience of vasomotor symptoms—hot flashes. An advertisement placed in the local newspaper, campus wide e-mail, and flyers were used to recruit women with hot flashes. Sixty-one qualified women were enrolled. Prior to the study, each participant was required to familiarize herself with study protocol and sign an informed consent document. Participation in this clinical study was completely voluntary.

Sixty-one female participants between the ages of 40 and 58 who were experiencing hot flashes were recruited for this study. Fifty-two women (85.2%) completed all three survey sessions (the initial survey and then two and six months later). Among the nine women who dropped out during the 6-month period, six did not respond to follow-up contacts made by phone and mail and were not available to schedule an appointment for subsequent study sessions, two women changed jobs and could not fit the study into their new work schedule, and one woman did not adhere to the protocol and was removed from the study.

The MVS survey was a structured interview conducted in person. The survey was administered at three times: at the initial session, and then two months and six months later. During each session, a trained surveyor read each question to the participant and marked her answers on the MVS survey form. The same surveyor conducted all interviews. Each completed MVS survey form was coded only with the participant's identification number.

All women enrolled in the study were asked to take the initial survey, and then take it again after two and six months. To assess the test-retest reliability, a sub-group of 20 women with hot flashes was randomly selected from the study participants to re-take the survey 14 days later. Since the symptoms of hot flashes can change over time, a time-course with longer intervals between sessions was chosen to assess whether the MVS survey can detect within-person changes in hot flashes symptoms that can develop over a six-month period of time. Two weeks before each subsequent session, a reminder letter was mailed to each participant followed by a phone call to set up a specific day and time that was convenient for the participant to retake the survey.

Women participating in this study responded only to the newly designed MVS survey because other subjective instruments (daily dairies and electronic even markers) provide only a very limited measure of hot flashes and could not be used as an adequate standard for the MVS survey with its multiple characteristics of hot flashes. Comparison of the MVS survey results with measures obtained with an objective instrument will be performed in a separate study.

The majority of the MVS survey questions addressed the dimensions of hot flashes that were likely to change over time (i.e., duration, frequency, timing, duration of one episode, intensity, bothersomeness). During the last (third) study session, 50 women had continued to experience hot flashes and two (2) women had stopped having hot flashes (Table 1). Thirteen participants (25%) had had hysterectomy. During the first session, 44% of the women reported that hot flashes were interfering with their daily activities (bothersomeness); this incidence declined during the second and third session to 36% and 26%, respectively. During the six-month-long study, 23 women in first session, 19 women in the second session, and 22 women in the third session, reported the use of hormone therapy for hot flashes (36-44% of the participants). Among participants taking hormone therapy, 1.9% of women in the first session, 3.8% of women in the second session, and 5.7% of women in the third session, reported no relief of their hot flashes. The use of non-hormone treatment for hot flashes increased between the first and last sessions, from 38% to 52%. A history of contraceptive use was reported by 85% in the first session, 92% in the second session, and 96% in the third session. The results of the initial survey from the 9 women who dropped from the study were comparable to those who stayed in the study.

TABLE 1
Characteristics of study participants.

| | Survey 1 | Survey 2 | Survey 3 |
|---|---|---|---|
| Number of women surveyed | 52 | 52 | 52 |
| Women with hot flashes (number) | 52 | 52 | 50 |
| Average age hot flashes started | 40.6 | 40.0 | 40.7 |
| (years) | | | |
| Age range at start of hot flashes | (27-54) | (27-52) | (33-53) |
| (years) | | | |

Number and percentage (in parenthesis) of women with:

| | | | |
|---|---|---|---|
| Hysterectomy | 13 (25%) | 13 (25%) | 13 (25%) |
| Bothersome hot flashes | 24 (44%) | 15 (36%) | 13 (26%) |
| Hormone therapy (HT) for hot | 23 (44%) | 19 (36%) | 22 (42%) |
| flashes | | | |
| Non-hormone treatment for hot | 20 (38%) | 23 (44.2%) | 27 (52%) |
| flashes | | | |
| History of contraceptive use | 44 (84.6) | 48 (92%) | 50 (96%) |

Average age of participants: 49.5 years; Age range: 40-58 years

The assessment of how well the MVS survey measured the symptoms of vasomotor hot flashes and its meaningfulness could only be based on face validity and content validity. The criterion validity analysis was performed due to the inability to identify an adequate reference instrument for the MVS survey.

The content of the MVS survey exclusively focused on various measures of hot flashes and their associated conditions. There was an initial set of 18 questions that addressed the specific dimensions of hot flashes, such as duration, frequency, timing, duration of one episode, intensity, bothersomeness (interference with quality of life), and age at their onset. The other 21 questions addressed issues associated with hot flashes that influence women's experience of menopausal or post-hysterectomy hot flashes. The content of these questions was related to hysterectomy, hormone therapy for hot flashes (treatment type, duration, effectiveness), non-hormonal therapy for hot flashes, and the use of contraceptive medications. All the MVS survey questions measured to a high degree (quantitatively and qualitatively) many of the specific aspects of hot flashes.

A newly developed questionnaire designed specifically for subjective assessment of hot flashes—the Menopausal or post-hysterectomy Vasomotor Symptoms (MVS) survey—was administered to all study participants. The MVS survey was designed to be short, simple, clear, easy to respond to, and easy to administer. The one-page form had 39 questions addressing the various dimensions of hot flashes and associated conditions (FIG. 3). Questions were mutually exclusive. Thirty-five survey questions were closed- response and required either a “Yes” or “No” answer (nominal measurements). For three of the 39 questions, the participant needed to provide a number reflecting the age or year when a specific hot-flash-associated event occurred (numerical measurements), and one question asked for the name of the hormonal preparation used to treat hot flashes. In the MVS survey, each specific dimension of hot flashes was addressed by a group of questions: duration of the period with hot flashes (questions 2-5), frequency (questions 6-8), timing—day or night (questions 9, 10), duration of one episode (questions 11-13), intensity (questions 14-16), and bothersomeness—quality of life (question 17). Questions within each group addressed the quantitative and qualitative measures of hot flashes. In addition to questions on various measures of hot flashes, the MVS survey included a set of questions about conditions associated with hot flashes, such as hysterectomy (questions 19, 20), hormone therapy and its type (questions 21-27), duration of hormone therapy (questions 28-32), effectiveness of hormone therapy in relieving hot flashes (questions 33-35), age hormone therapy was started (question 36), non-hormonal therapies for hot flashes (question 37), and history of use of hormonal contraceptives (questions 38-39). If a woman did not remember the name of a medication, she was asked to identify her medication on a color chart from the Physician's Desk Reference (PDR) with currently available hormonal preparations (question 23). If she could not recognize the hormonal preparation on the chart, she was asked to provide the dosage form (pill, patch, cream or injection) of the drug (questions 24-27). Reponses to survey questions were marked in the “Answer” column. A separate column on the survey form (“Notes”) was designated for additional comments to be written by the person conducting the survey.

In terms of face validity, the results of the MVS survey had the appearance of truth and reality. In terms of content validity, the MVS survey questions fulfilled, to a high degree, many specific details related to women's experience of hot flashes. Thus, the questions chosen for the MVS survey were representative of the concepts they were intended to reflect.

Fifteen items (questions) that were normally distributed and covariated with one another were factor-analyzed with both orthogonal and oblique rotation in order to identify item clusters. As is shown in Table 2, four factors emerged and 14 from the original 15 items were retained. The four factors were named: “Life Interference,” “Acute Experience,” “Mix of Acute and Chronic Experience,” and “Chronic Experience.” The amount of common variance accounted for by the four factors was 17.8%, 14.3%, 12.6%, and 11.7% respectively. Table 2 shows the factor loadings associated with each item. The factor loadings generated with Varimax rotation were virtually identical with loadings that were generated with Direct Oblimin rotation. Thus, orthogonal loadings were reported here. One question, “Do you feel drenched with sweat during your hot flashes?”, did not load highly on any of the factors, nor did it account for another unique factor.

TABLE 2
Factor analysis of select MVS survey items.

| Factor Loading | The MVS survey item (question) |
|---|---|
| Factor 1: Life Interference | |
| −0.76 | Less than 5 hot flashes per 24 hrs |
| 0.61 | Hot flashes at night |
| 0.40 | Feel mildly sweaty during hot flashes |
| 0.68 | Hot flashes interrupt daily activities |
| Factor 2: Acute | |
| Experience | |
| 0.68 | Have had hot flashes for 6 to 12 months |
| 0.77 | Hot flashes last less than 2 minutes |
| 0.88 | Hot flashes last 2 to 5 minutes |
| Factor 3: Mix of Acute | |
| and Chronic Experience | |
| 0.48 | Have had hot flashes for less than 6 months |
| −0.66 | Have had hot flashes for 1 to 2 years |
| 0.40 | Have had hot flashes during day |
| 0.68 | Hot flashes last longer than 5 minutes |
| Factor 4: Chronic | |
| Experience | |
| 0.74 | Have hot flashes for more than 2 years |
| −0.49 | Have hot flashes 5 to 10 times per 24 hrs |
| 0.56 | Have more than 10 hot flashes per 24 hrs |

Positive and negative factor loadings indicate more “yes” or “no” responses to that item, respectively.

Eleven questions from the MVS survey (questions 3, 4, 5, 6, 7, 8, 11, 13, 14, 15, 17) were appropriate for an internal consistency analysis using the Kuder-Richardson 20 (KU-20) coefficient for dichotomous data. The value of the KU-20 coefficient was α=0.87, indicating high internal consistency among the items.

The MVS was evaluated for its test-retest reliability by estimating Pearson's r coefficients for datasets collected from re-administering the MVS survey to a randomly selected group (N=20) of women 24 to 48 hours and 14 days after the initial session (baseline). The values of the Pearson's correlation coefficients served as an index for the consistency of answers provided during the two re-tests administered within a short period of time. Twelve women were available to provide re-test answers 24 to 48 hours after the initial survey, and 20 women were re-tested 14 days later. After 24 to 48 hours, the test-retest correlation coefficients varied between 1.000 and 0.837 and were highly significant for all survey questions (p≦0.001). After 14 days, the test-retest correlations for the majority of the questions varied between 1.000 (p=0.000) and 0.798 (p=0.001); only two questions had a lower correlation of 0.577 (p=0.008).

During survey evaluation, intra-rater reliability was not assessed because the same individual administered the MVS at all sessions, and moreover, the simple and very specific answers assured a high consistency of responses. The MVS was evaluated for its test-retest reliability. The MVS survey was re-administered to a randomly selected group (n=20) of women 14 days after the initial session. The results of the test-retest reliability, computed using Pearson's r coefficients, are presented in Table 3. After 14 days, the test-retest correlations for the majority of the questions varied between 1.000 (p=0.000) and 0.80 (p=0.001); three questions had a correlation of 0.58 (p=0.008).

TABLE 3
Test-retest reliability of the MVS survey.

After
14 days
(n = 20)

| | | |
|---|---|---|
| Dimension of hot flashes | r* | p value |

Duration

| | | |
|---|---|---|
| <6 months | 1.000 | 0.000 |
| 7-12 months | 1.000 | 0.000 |
| 13 months-2 years | 1.000 | 0.000 |
| >2 years | 1.000 | 0.000 |

Frequency

| | | |
|---|---|---|
| <5/24 hours | 0.798 | 0.001 |
| 5-10/24 hours | 1.000 | 0.000 |
| >10/24 hours | 0.899 | 0.001 |

Timing

| | | |
|---|---|---|
| Day | 0.899 | 0.001 |
| Night | 0.899 | 0.001 |

Duration of one episode

| | | |
|---|---|---|
| <2 minutes | 0.577 | 0.008 |
| 2-5 minutes | 0.798 | 0.001 |
| >5 minutes | 1.000 | 0.000 |

Intensity

| | | |
|---|---|---|
| Felt drenched | 0.798 | 0.001 |
| Felt mildly sweaty | 0.577 | 0.008 |

Bothersomeness

| | | |
|---|---|---|
| Interrupted daily activities | 1.000 | 0.000 |
| Age hot flashes started | | |
| Age | 0.798 | 0.001 |

Hysterectomy

| | | |
|---|---|---|
| Surgery | 1.000 | 0.000 |
| Age | 0.789 | 0.001 |

Hormone therapy

| | | |
|---|---|---|
| Used | 0.899 | 0.001 |

Duration of hormone therapy

| | | |
|---|---|---|
| <6 months | 0.798 | 0.001 |
| 6-12 months | 0.899 | 0.001 |
| 1-2 years | 1.000 | 0.000 |
| 2-10 years | 1.000 | 0.000 |
| >10 years | 1.000 | 0.000 |

Relief from hot flashes after hormone therapy

| | | |
|---|---|---|
| Complete | 1.000 | 0.000 |
| Partial | 0.798 | 0.001 |
| None | 0.577 | 0.008 |

Age hormone therapy started

| | | |
|---|---|---|
| Age | 0.798 | 0.001 |

Herbal preparations for hot flashes

| | | |
|---|---|---|
| Used | 0.899 | 0.001 |

Contraceptives

| | | |
|---|---|---|
| Used | 1.000 | 0.000 |

*Pearson's r correlation coefficient

During survey evaluation, intra-rater reliability was not assessed because the same individual administered the MVS at all sessions. The simple and very specific answers evidenced a high consistency of responses.

Many dimensions of hot flashes change over time. Pearson's correlation coefficients were estimated for responses obtained at three different periods of time: six months (responses obtained during first and third surveys), four months (responses obtained during second and third surveys), and two months (responses obtained during first and second surveys). The Pearson's correlation coefficient values were estimated for each survey question in order to assess time-related changes in various dimensions of hot flashes.

By administering the MVS survey three times over six months, the same measurements were repeatedly made of the same participant to determine if the MVS survey can detect changes in hot flashes. There were shifts in “Yes” and “No” answers in specific quantitative measures of hot flashes during the three survey sessions. Due to the nature of hot flashes, responses to some of the survey questions were expected to change/fluctuate over time, while other answers were not expected to change. Responses to the MVS survey provided at each of the three survey sessions during six-month period were used to establish the Pearson's correlation coefficients. These correlation coefficients were obtained for measures taken for time intervals of two months (between sessions one and two), four months (between sessions two and three), and six months (between sessions one and three). The values of Pearson's correlation coefficients seem to reflect the stability (sensitivity to change over time) of a specific dimension of hot flashes as well as the consistency of answers, and consequently, assisted in identifying three categories of MVS survey questions. The first category—the MVS survey questions on hot flash symptoms that were expected to change, i.e., duration, frequency, timing, duration of one episode, intensity, bothersomeness—was represented by very low Pearson's coefficients values (<0.700). The second category—the MVS survey questions not directly related to a specific dimension of hot flashes, for example, the extent of relief after hormone therapy—was represented by both low (for harder to discern options) and high (for options that were more clear-cut) values of the Pearson's coefficient. The third category—the MVS survey questions for which answers were very unlikely to change, for example age, history use of hormone therapy or history hysterectomy—had high (>0.700) values of Pearson's correlation coefficient. High correlation coefficients that measure consistently for periods of two, four, and six months imply that the specific measures assessed with the MVS survey did not change. Pearson's correlation coefficients estimated at two, four, and six months for measures of hot flashes expected to change over time were consistently low. Inconsistent over-time values of Pearson's coefficients were observed for questions that used a more ambiguous and detailed measure that was hard to discern consistently by the participants.

An additional approach to addressing the sensitivity of the MVS survey to changes in hot flashes was based on comparing the MVS survey results obtained during first and last sessions (6 months apart) for a sub-group of women who voluntarily reported (just before re-taking the survey) a change in their hot flashes and a sub-group of women who only provided responses to the survey. During the last survey, eighteen women (36%) provided unsolicited comments about experiencing changes in their hot flashes symptoms. Responses to 12 questions from the MVS survey related to the symptoms of hot flashes that were likely to change over time were used for this evaluation. Each sub-group of 10 women was randomly selected by analyzing the notes made by the surveyor on the individual MVS survey forms. No significant differences were found between these two randomly selected sub-groups of women with (Table 4). The Mann-Whitney analysis showed no significant differences between these two groups; for all 12 evaluated questions, the P values were in a range from 0.247 to 1.000 (data not shown). These results lead up to accept the null hypothesis that there was no difference between the MVS survey results of women who independent from the survey question, reported changes in hot flashes and women who did not make any comments in addition to survey answers. The outcome of the analysis of the survey results strongly suggested that the MVS survey was sensitive to changes in symptoms of hot flashes during a period of six months.

By administering the MVS survey three times over six months, the same measurements were repeatedly made in the same participant to determine if the MVS survey can detect changes in hot flashes. The number of “Yes” and “No” answers recorded in the first, second, and third surveys is presented in Table 4. During the 6 months, there were shifts in “Yes” and “No” answers mainly for questions that addressed quantitative measures of specific dimensions of hot flashes during the three survey sessions.

TABLE 4
Profile of responses to the MVS survey at three different study sessions and the
Pearson's correlation coefficients among surveys.

| | | | | | | |
|---|---|---|---|---|---|---|
| Domain of | Survey 1 | Survey 2 | Survey 3 | Surveys | Surveys | Surveys |
| Hot flashes | (Baseline) | (+2 months) | (+6 months) | 1 vs. 2 | 2 vs. 3 | 1 vs. 3 |

Duration

| | | | | | | |
|---|---|---|---|---|---|---|
| <6 months | Y = 8 N = 42 | Y = 4 N = 46 | Y = 0 N = 50 | 0.475** | NC | NC |
| 7-12 months | Y = 13 N = 37 | Y = 9 N = 41 | Y = 5 N = 45 | 0.179 | 0.191 | −0.198 |
| 13 months-2 years | Y = 13 N = 37 | Y = 18 N = 32 | Y = 2 N = 39 | 0.315* | 0.290* | −0.042 |
| >2 years | Y = 16 N = 34 | Y = 19 N = 31 | Y = 4 N = 26 | 0.700** | 0.567** | 0.542** |

Frequency

| | | | | | | |
|---|---|---|---|---|---|---|
| <5/24 hrs | Y = 29 N = 21 | Y = 30 N = 20 | Y = 27 N = 23 | 0.546** | 0.475** | 0.434** |
| 5-10/24 hrs | Y = 17 N = 33 | Y = 16 N = 34 | Y = 19 N = 31 | 0.413** | 0.346* | 0.395** |
| >10/24 hrs | Y = 7 N = 43 | Y = 5 N = 45 | Y = 4 N = 46 | 0.442** | −.393** | 0.518** |

Timing

| | | | | | | |
|---|---|---|---|---|---|---|
| Day | Y = 45 N = 5 | Y = 44 N = 6 | Y = 46 N = 4 | 0.492** | 0.572** | 0.393** |
| Night | Y = 48 N = 2 | Y = 45 N = 5 | Y = 46 N = 4 | 0.612** | 0.393** | −0.06 |

Duration of one episode

| | | | | | | |
|---|---|---|---|---|---|---|
| <2 minutes | Y = 23 N = 27 | Y = 21 N = 29 | Y = 22 N = 28 | 0.190 | 0.307* | 0.233 |
| 2-5 minutes | Y = 19 N = 31 | Y = 23 N = 27 | Y = 25 N = 25 | 0.104 | 0.201 | 0.124 |
| >5 minutes | Y = 8 N = 42 | Y = 6 N = 44 | Y = 3 N = 47 | 0.342* | −0.093 | 0.119 |

Intensity

| | | | | | | |
|---|---|---|---|---|---|---|
| Felt drenched | Y = 23 N = 27 | Y = 22 N = 28 | Y = 24 N = 26 | 0.556** | 0.600** | 0.559** |
| Felt mildly sweaty | Y = 37 N = 13 | Y = 32 N = 18 | Y = 33 N = 17 | 0.220 | 0.253 | 0.441** |

Bothersomeness

| | | | | | | |
|---|---|---|---|---|---|---|
| Interrupted daily | Y = 24 N = 26 | Y = 15 N = 35 | Y = 13 N = 37 | 0.507** | 0.507** | 0.252 |
| activities | | | | | | |

Age hot flashes started

| | | | | | | |
|---|---|---|---|---|---|---|
| Age (years, average) | 45.9 | 46.0 | 45.7 | 0.827** | 0.896** | 0.703** |

Hysterectomy

| | | | | | | |
|---|---|---|---|---|---|---|
| Surgery | Y = 13 N = 39 | Y = 13 N = 39 | Y = 13 N = 39 | 1.000** | 1.000** | 1.000** |
| Age (years, average) | 40.6 | 40.0 | 40.7 | 0.961** | 0.989** | 0.977** |

Hormone therapy

| | | | | | | |
|---|---|---|---|---|---|---|
| Used | Y = 23 N = 29 | Y = 19 N = 33 | Y = 22 N = 30 | 0.852** | 0.805** | 0.962** |
| Type | E = 7 P = 5 | E = 7 P = 3 | E = 5 P = 4 | 0.787** | 0.723** | NC |
| | E + P = 6 | E + P = 8 | E + P = 9 | | | |

Duration of hormone therapy

| | | | | | | |
|---|---|---|---|---|---|---|
| <6 months | Y = 1 N = 15 | Y = 1 N = 17 | Y = 2 N = 17 | 1.000** | 0.686** | NC |
| 6-12 months | Y = 0 N = 16 | Y = 1 N = 17 | Y = 0 N = 18 | NC | NC | NC |
| 1-2 years | Y = 3 N = 13 | Y = 7 N = 11 | Y = 4 N = 14 | 0.289 | 0.396 | −0.25 |
| 2-10 years | Y = 9 N = 7 | Y = 6 N = 12 | Y = 9 N = 9 | 0.423 | 0.471** | 0.167 |
| >10 years | Y = 3 N = 13 | Y = 3 N = 15 | Y = 3 N = 15 | 1.000** | 1.000** | 1.000** |

Relief from hot flashes after hormone therapy

| | | | | | | |
|---|---|---|---|---|---|---|
| Complete | Y = 8 N = 8 | Y = 7 N = 11 | Y = 7 N = 11 | 0.775** | 0.766** | 0.408 |
| Partial | Y = 8 N = 8 | Y = 9 N = 9 | Y = 8 N = 10 | 0.882** | 0.671** | 1.000** |
| None | Y = 1 N = 15 | Y = 2 N = 16 | Y = 3 N = 15 | 1.000** | 0.791** | NC |

Age hormone therapy started

| | | | | | | |
|---|---|---|---|---|---|---|
| Age (years, average) | 40.8 | 43.7 | 43.9 | 0.937* | 0.956** | 0.960** |

Herbal preparations for hot flashes

| | | | | | | |
|---|---|---|---|---|---|---|
| Used | Y = 20 N = 32 | Y = 23 N = 29 | Y = 27 N = 25 | 0.569** | 0.623** | 0.602** |

Contraceptives

| | | | | | | |
|---|---|---|---|---|---|---|
| Used | Y = 44 N = 8 | Y = 48 N = 4 | Y = 50 N = 2 | 0.677** | 0.693** | 0.469** |
| Duration (years, | 8.5 | 7.9 | 9.1 | 0.638** | 0.591** | 0.810** |
| average) | | | | | | |

Y = yes;
N = no;
E = estrogens;
P = progestins;
NC = not computed;
*= p < 0.05;
**= p < 0.01

Responses to the MVS survey provided at each of the three survey sessions were used to establish the Pearson's correlation coefficients (Table 5). These correlation coefficients were obtained for measures taken for time intervals of two months (between sessions one and two), four months (between sessions two and three), and six months (between sessions one and three). The values of Pearson's r correlation coefficients reflect the sensitivity to change over time of a specific dimension of hot flashes as well as the consistency of answers, and consequently, assisted in identifying three categories of MVS survey questions. The first category—the MVS survey questions on hot flash symptoms that were expected to change, i.e., duration, frequency, timing, duration of one episode, intensity, bothersomeness—was represented by <0.700 Pearson's r coefficients values. The second category—the MVS survey questions not directly related to a specific dimension of hot flashes, for example, the extent of relief after hormone therapy—was represented by both lower (for harder to discern options) and higher (for options that were more clear-cut) values of the Pearson's r coefficient. The third category—the MVS survey questions for which answers were very unlikely to change, for example age, history use of hormone therapy or history of hysterectomy—had high (>0.70) values of Pearson's r correlation coefficient. High correlation coefficients measured consistently for periods of two, four, and six months imply that the specific measures assessed with the MVS survey did not change. Pearson's r correlation coefficients estimated at two, four, and six months for measures of hot flashes expected to change over time were consistently low. Inconsistent “over-time” values of Pearson's coefficients were observed for questions that addressed a specific quantitative measure that either was vulnerable to change over time or hard to discern consistently by the participants.

TABLE 5

| Dimension of hot flashes | Pearson Chi-Square value | P value |
|---|---|---|
| Frequency | 0.000 | 1.000 |
| <5/24 hours | 1.077 | 0.584 |
| 5-10/24 hours | 1.067 | 0.587 |
| >10/24 hours | 2.000 | 0.368 |
| Timing | 0.000 | 1.000 |
| Day | 0.392 | 0.531 |
| Night | 1.053 | 0.305 |
| Duration of one episode | 0.000 | 1.000 |
| <2 minutes | 3.333 | 0.189 |
| 2-5 minutes | 0.800 | 0.670 |
| >5 minutes | 0.410 | 0.815 |
| Intensity | 0.879 | 0.348 |
| Felt drenched | 4.143 | 0.126 |
| Felt mildly sweaty | 1.067 | 0.587 |
| Felt hot | NC | — |
| Bothersomeness | 0.208 | 0.648 |
| Interrupted daily activities | 3.533 | 0.171 |

NC = not computed

The results from the validity and reliability analysis and the in-person experience with administering the survey provided a valuable basis for improvements in the MVS survey. Amendments to the original version of the MVS survey were made by 1) clarifying the wording of questions, 2) eliminating ambiguous and quantitatively small measures, 3) removing questions that consistently did not result in a meaningful answer, and 4) standardizing and clarifying the recording of answers to the survey questions. In questions on hot flash frequency, “per day” was changed to “in 24 hours” (questions 6-7). Also, “Do you take hormones” in question 21, was changed to “Have you ever taken hormones.” Less consistency in answers was observed for questions that asked for details that were not always possible to perceive and discern clearly. For example, in the options for the duration of hot flashes and duration of hormone treatment, it seemed harder for respondents to consistently choose between “less than 6 months” and “7 to 12 months,” or for terms of frequency, “less than 5 per 24 hours” and “5-10 per 24 hours.” The question that asked for the identification of hormonal medication on a chart and questions on dosage forms were removed because they were less relevant and their consistency was affected by the variability in hormonal preparations available on the market and the fact that during therapy, women often switched to a different hormonal preparation. The main intent for inclusion of these questions in the original version of the MVS survey was to help a woman identify a hormonal preparation if she did not know the drug's name. Nine questions were removed from the original survey. The amended MVS survey has a total of 30 questions (FIG. 4).

### REFERENCES

U.S. Census Bureau. Projected population of the United States, by Age and Sex: 200 to 2050. www.census.gov/ipc/www/usinterimproj/ Internet release date: Mar. 18, 2004.
Kronenberg, F. Hot flashes: Epidemiology and physiology. Ann NY Acad Sci 1990;592:52-86.
McKinlay, S M, Brambilla, D J, Posner, J G. The normal menopause transition. Maturitas 1992;14:103-115.
Jaszman, J, van Lith, W D, Zaat, J C A. The perimenopause: the statistical analysis of a survey: Part A. Medical Gynecologic Sociology 1969;4:268-277.
McKinlay, S M, Jeffreys, M. The menopausal syndrome. Br J Prevent Med. 1974;28:108-111.
Hunter, M S, Liao, K L M. A psychological analysis of menopausal hot flashes. Br J Clin Psych 1995;34:589-599.
Utian, W H. Current status of menopause and postmenopausal estrogen therapy. Obst. Gynecol Survey 1977;32:193-197.
Stearns, V, Ullmer, L, Lopez, J F, Smith, Y, Isaacs, C, Hayes, D. Hot flashes. Lancet 2002;360:1851-1861.
Grisso, J W, Freeman, E W, Maurin, E, Garcia-Espana, B, Berlin, J. Racial differences in menopause information and the experience of hot flashes. J Gen Intern Med 1999; 14:98-103.
Gold, E B, Stemfeld, B, Kelsey, J L, Brown, C, Mouton, C, Reame, N, Salamone, L. Relation of demographic and lifestyle factors to symptoms in a multi-racial/ethnic population of women 40-55 years of age. Am J Epidemiol 2000;152:463-73.
Freedman, R R. Biochemical, metabolic, and vascular mechanisms in menopausal hot flashes. Fert Steril 1998;70:332-337.
Blatt, M H, Weisbader, H, Kupperman, H S. Vitamin E and climacteric syndrome: a failure of effective control as measured by menopausal index. AMA Arch Intern Med 1953;91:792-799.
http://www.mapi-research-inst.org (Distribution of Health Outcome Instruments)
Greene, J G. Constructing a standard climacteric scale. Maturitas 1998; 29:25-31.
http://www.progolid.org/public
Heinemann, K, Ruebig, A, Potthoff, P, Schneider H P G, Sterlow, F, Heinemann L A J, Thai D M. The Menopause Rating Scale (MRS): A methodological review. Health and Quality of Life Outcomes, 2004;2:45-53.
Carpenter, J S, Andrykowski, M A, Freedman, R R, Munn, R. Feasibility and psychometrics of an ambulatory hot flash monitoring device. Menopause 1999;6:209-215.
Carpenter, J S, Monahan, P O, Azzouz, F. Accuracy of subjective hot flush reports compared with continuous external skin conductance monitoring. Obstet Gynecol 2004;104:1322-1326.
Aday, L A., Designing and conducting health surveys, 2nd ed. San Francisco, Calif., Jossey-Bass, John Wiley & Sons, Inc., 1996.
Lacity, M and Jansen, M A. Understanding qualitative data: A framework of text analysis methods. J Management Information System 1994; 11:137-160.
Polinghorne, D E (1988). Narrative knowing and the human sciences. Albany: State University of New York Press.
Tabachnick, B G, & Fidell, L S.Using Multivariate Statistics, 4th ed. Needham Heights: Allyn and Bacon, 2001.
Alder, E. The Blatt-Kupperman menopausal index: a critique. Maturitas 1998;29:19-24.
Tice, J A, Ettinger, B, Ensrud, K, Wallace, R, Blackwell, T, Cummings, S. Phytoestrogen supplements for treatment of hot flashes: the isoflavone clover extract (ICE) study. A randomized controlled trial. JAMA 2003;290:207-214.
Cagnacci, A, Melis, B G, Soldani, R., Paoletti, A M, Gambacciani M, Spinetti, A, Fioretti, P. Neuroendocrine and clinical effects of transdermal 17beta-estradiol in postmenopausal women. Maturitas 1991; 13:283-296.
Archer, D F, Fischer, L A, Rich, D, Schade, G H, Schwartz, S, Wittcoff, H, Clark, C S, Maloney, K, Smith, F O. Estrace vs. Premarin for treatment of menopausal symptoms: dosage comparison study. Advances in Therapy 1992;9:21-31.
Avis, N E, Crawford S, McKinlay, S M. Psychosocial, behavioral, and health factors related to menopause symptomatology. Women's Health: Research on Gender, Behavior, and Policy 1997;3:103-120.
Im, E-O, Chee, W, Bender, M, Cheng, C-Y, Tsai, H-M, Kang, N M, Lee, H. The psychometric properties of pen-and-pencil and internet versions of the midlife women's symptom index (MSI). Intern J Nurs Studies 2005;42:167-177.

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. Although preferred embodiments of the present invention have been described in detail, it will be understood by those skilled in the art that various modifications can be made therein without departing from the spirit and scope of the invention as set forth in the appended claims.

What is claimed is:

1. A method of assessing a person's menopausal or post-hysterectomy symptoms comprising the steps of:
providing a first set and a second set of questions to the person wherein the first set of questions comprise four or more questions directed to one or more physical conditions of the person and the second set of questions comprise two or more questions directed to one or more medical conditions of the person;
receiving answers to the first set and second set of questions; and
assessing the person's menopausal or post-hysterectomy symptoms based on the received answers.

2. The method as recited in claim 1, wherein the menopausal or post-hysterectomy symptoms comprise hot flashes.

3. The method as recited in claim 1, wherein the physical conditions comprise:
a duration of the menopausal or post-hysterectomy symptoms;
a frequency of the menopausal or post-hysterectomy symptoms;
an episode duration of the menopausal or post-hysterectomy symptoms;
an intensity of the menopausal or post-hysterectomy symptoms;
a bothersomeness of the menopausal or post-hysterectomy symptoms; and
an onset age of the menopausal or post-hysterectomy symptoms.

4. The method as recited in claim 1, wherein the medical conditions comprise:
a hysterectomy;
a hormone therapy;
a non-hormonal therapy; and
a contraception type.

5. The method as recited in claim 1, wherein the first set of questions comprise:
one or more questions regarding how long the person has had hot flashes;
one or more questions regarding how many hot flashes the person has during a day;
one or more questions regarding when the person has most of the hot flashes in during a day;
one or more questions regarding how long the hot flashes last;
one or more questions regarding sweating during the hot flashes;
one or more questions regarding interrupted daily activities or sleep; and
one or more questions regarding an age when the person started having hot flashes.

6. The method as recited in claim 1, wherein the second set of questions comprise:
one or more questions regarding a hysterectomy;
one or more questions regarding hormone therapy;
one or more questions regarding herbal preparations; and
one or more questions regarding contraceptive medications.

7. The method as recited in claim 1, wherein substantially all of the first set and second set of questions are closed-response questions.

8. The method as recited in claim 1, wherein substantially all of the answers comprise a yes, a no or a not applicable.

9. The method as recited in claim 1, wherein the step of assessing the person's menopausal or post-hysterectomy symptoms comprises comparing the received answers to one or more diagnostic profiles, one or more baseline criteria, a stored library of previously received answers and corresponding patient profiles.

10. The method as recited in claim 1, further comprising the step of providing the assessment to the person.

11. The method as recited in claim 1, further comprising the step storing the answers to the first and second sets of questions in a database.

12. The method as recited in claim 11, further comprising the step of providing access to the database to one or more medical entities.

13. The method as recited in claim 12, wherein the one or more medical entities comprise a hospital, a clinic, a doctor's office, an educational facility, a governmental facility, a doctor or a researcher.

14. The method as recited in claim 1, further comprising the steps of:
providing a third set of questions to the person wherein the third set of questions are directed to creating a profile for the person; and
creating the profile for the person.

15. The method as recited in claim 1, further comprising the steps of:
receiving one or more diary entries from the person; and
storing the one more diary entries.

16. The method as recited in claim 1, further comprising the step of verifying an identity of the person.

17. The method as recited in claim 1, further comprising the step of determining a treatment for the menopausal or post-hysterectomy symptoms based on the assessment.

18. The method as recited in claim 1, further comprising the steps of:
receiving medical information about the person from a health care provider; and
storing the medical information.

19. The method as recited in claim 18, wherein the health care provider comprises a doctor, a nurse, a physician's assistant, a researcher or a medical technician.

20. The method as recited in claim 1, further comprising the step of reassessing the person's menopausal or post-hysterectomy symptoms after a specified time period.

21. The method as recited in claim 20, wherein the specified time period comprises approximately fourteen days, two months, six months or a time based on the assessment.

22. The method as recited in claim 20, wherein the step of reassessing the person's menopausal or post-hysterectomy symptoms comprises the steps of:
providing the first set and the second set of questions to the person after the specified time period;
receiving new answers to the first set and second set of questions; and
reassessing the person's menopausal or post-hysterectomy symptoms based on the received new answers and the received answers.

23. The method as recited in claim 20, further comprising the step of evaluating an effectiveness of a treatment for the menopausal or post-hysterectomy symptoms based on the reassessment.

24. The method as recited in claim 1, wherein the steps of providing the first set and the second set of questions to the person and receiving the answers to the first set and second set of questions are performed on one or more computers.

25. The method as recited in claim 24, wherein the one or more computers comprise a personal computer, a laptop computer, a personal data assistant, a telephone, a mobile telephone or a wireless communication device.

26. A computer program embodied on a computer readable medium for assessing a person's menopausal or post-hysterectomy symptoms comprising:
a code segment for providing a first set and a second set of questions to the person wherein the first set of questions comprise four or more questions directed to one or more physical conditions of the person and the second set of questions comprise two or more questions directed to one or more medical conditions;
a code segment for receiving answers to the first set and second set of questions; and
a code segment for assessing the person's menopausal or post-hysterectomy symptoms based on the received answers.

27. A system for assessing a person's menopausal or post-hysterectomy symptoms comprising:
a computer that provides a first set and a second set of questions to the person and receives one or more answers to the first set and second set of questions, wherein the first set of questions comprise four or more questions directed to one or more physical conditions of the person and the second set of questions comprise two or more questions directed to one or more medical conditions such that the one or more answers to the first set and second set of questions are used to assess the person's menopausal or post-hysterectomy symptoms; and
a database communicably coupled to the computer, the database storing the first set and the second set of questions and the one or more answers to the first set and second set of questions.

28. The system as recited in claim 27, wherein the menopausal or post-hysterectomy symptoms comprise hot flashes.

29. The system as recited in claim 27, wherein the physical conditions comprise:
a duration of the menopausal or post-hysterectomy symptoms;
a frequency of the menopausal or post-hysterectomy symptoms;
an episode duration of the menopausal or post-hysterectomy symptoms;
an intensity of the menopausal or post-hysterectomy symptoms;
a bothersomeness of the menopausal or post-hysterectomy symptoms; and
an onset age of the menopausal or post-hysterectomy symptoms.

30. The system as recited in claim 27, wherein the medical conditions comprise:
a hysterectomy;
a hormone therapy;
a non-hormonal therapy; and
a contraception type.

31. The system as recited in claim 27, wherein the first set of questions comprise:
one or more questions regarding how long the person has had hot flashes;
one or more questions regarding how many hot flashes the person has during a day;
one or more questions regarding when the person has most of the hot flashes in during a day;
one or more questions regarding how long the hot flashes last;
one or more questions regarding sweating during the hot flashes;
one or more questions regarding interrupted daily activities or sleep; and
one or more questions regarding an age when the person started having hot flashes.

32. The system as recited in claim 27, wherein the second set of questions comprise:
one or more questions regarding a hysterectomy;
one or more questions regarding hormone therapy;
one or more questions regarding herbal preparations; and
one or more questions regarding contraceptive medications.

33. The system as recited in claim 27, wherein substantially all of the first and second sets of questions are closed-response questions.

34. The system as recited in claim 27, wherein substantially all of the answers comprise a yes, a no or a not applicable.

35. The system as recited in claim 27, wherein the person's menopausal or post-hysterectomy symptoms are reassessed after a specified time period.

36. The system as recited in claim 35, wherein the specified time period comprises approximately fourteen days, two months or six months.

37. The system as recited in claim 35, wherein the reassessment is performed by providing the first set and the second set of questions to the person after the specified time period, receiving the answers to the first and second sets of questions and assessing the person's menopausal or post-hysterectomy symptoms based on the received answers.

38. The system as recited in claim 27, wherein a treatment is determined for the menopausal or post-hysterectomy symptoms based on the assessment.

39. The system as recited in claim 27, wherein effectiveness of a treatment for the menopausal or post-hysterectomy symptoms is evaluated based on the assessment.

40. The system as recited in claim 27, further comprising:
a communications network communicably coupled to the computer; and
one or more additional computers communicable coupled to the network that provide access to the database to one or more medical entities.

41. The system as recited in claim 40, wherein the one or more medical entities comprise a hospital, a clinic, a doctor's office, an educational facility, a governmental medical facility, a doctor or a researcher.

42. The system as recited in claim 40, wherein the computer or the one or more additional computers comprise a personal computer, a laptop computer, a personal data assistant, a telephone, a mobile telephone or a wireless communication device.

43. A survey for assessing a person's menopausal or post-hysterectomy symptoms comprising a set of questions as substantially shown in FIG. 3 or FIG. 4.
